(19) United States
(12) Patent Application Publication
(10) Pub. No.: US 2018/0312902 A1
(43) Pub. Date: Nov. 1, 2018

(54) NUCLEIC ACID PURIFICATION DEVICE AND NUCLEIC ACID PURIFICATION METHOD

(71) Applicant: UNIST(ULSAN NATIONAL INSTITUTE OF SCIENCE AND TECHNOLOGY), Ulsan (KR)

(72) Inventors: Yoon Kyoung CHO, Ulsan (KR); Chi Ju KIM, Ulsan (KR); Tae Hyeong KIM, Ulsan (KR)

(21) Appl. No.: 15/770,238

(22) PCT Filed: Oct. 21, 2016
(86) PCT No.: PCT/KR2016/011902

§ 371 Date: Apr. 23, 2018

(30) Foreign Application Priority Data
Oct. 22, 2015 (KR) 10-2015-0147565

(51) Int. Cl. C12Q 1/6806

(52) U.S. Cl. CPC C12Q 1/6806

(57) ABSTRACT

Provided is a nucleic acid purification device which is rotatably installed such that the entire processes of purifying a cell-free nucleic acid from a large amount of blood and a large amount of body fluid are integrated, the nucleic acid purification device including a disk in which a fluid is transferred by a centrifugal force, a supply portion installed in the disk and configured to supply a specimen and a reagent required for nucleic acid purification, an adsorption reaction chamber which is installed in the disk and is connected with the supply portion and in which an adsorption medium for adsorbing a nucleic acid is accommodated and the nucleic acid is adsorbed from the specimen, and a solution accommodating portion which is connected with an output side of an adsorption reaction portion along a centrifugal direction of the disk and in which a solution discharged through the adsorption reaction chamber by the centrifugal force is accommodated and is discharged to the outside.

### TECHNICAL FIELD

The present invention relates to a nucleic acid purification device and a nucleic acid purification method in which the entire nucleic acid (DNA) purification process for molecular diagnosis is integrated.

### BACKGROUND ART

An example of a field requiring a rapid and accurate nucleic acid purification technology is a molecular diagnosis field using a cell-free nucleic acid.

The cell-free nucleic acid (DNA) refers to a nucleic acid which escapes from a cell due to apoptosis, cell necrosis, and secretion, and exists in a bloodstream and a body fluid.

Physical characteristics of the cell-free nucleic acid, known to date, are that due to DNase action in blood and a body fluid, the length of the cell-free nucleic acid is averagely 200 base pairs, which is short, and the half life of the cell-free nucleic acid is in a range of 4 minutes to 30 minutes, which is unstable.

In recent years, with development of molecular diagnosis technologies such as next generation sequencing and digital polymer chain reaction (PCR), clinical researches using the cell-free nucleic acid have increased.

In particular, a diagnostic method in which as noninvasive sample collection (blood sampling), a fetal-derived cell-free nucleic acid is purified, analyzed, and diagnosed from mother's blood has been widely used in a prenatal diagnosis field in which a disease is determined and diagnosed before a fetus is born. Even in a cancer diagnosis field, researches in which a cell-free nucleic acid is purified from patient's blood and clinical significance is found using the same have been actively carried out.

The conventional method of purifying a cell-free nucleic acid is a method of collecting a certain amount of blood and a certain amount of body fluid, and manually purifying a nucleic acid using a commercialized kit by a skilled expert.

However, since this method requires four hours or more and an expert from blood sampling to nucleic acid purification, this method is a technology that is unsuitable for purifying a high-purity cell-free nucleic acid for molecular diagnosis.

Many researchers have been trying to automate nucleic acid purification using a lap-on-a-chip in which various functions performed in a laboratory are automated and downsized.

However, in most researches, due to nonexistence of a valve technology capable of adjusting fluid in a microfluidic chip, a chip assembling technology, and the like, a large number of sample processing modules necessarily required for cell-free nucleic acid purification may not be integrated, and only some of them are automated.

### DISCLOSURE

### Technical Problem

An exemplary embodiment of the present invention provides a fully automated nucleic acid purification device and a nucleic acid purification method in which the entire processes of purifying a cell-free nucleic acid from a large amount of blood and a large amount of body fluid are integrated using a centrifugal force-based disc-shaped chip.

Another embodiment of the present invention provides an integrated nucleic acid purification device and a nucleic acid purification method in which the entire processes of purifying a nucleic acid from various biofluids may be performed in an integrated manner using a centrifugal force-based microfluidic system.

### Technical Solution

A nucleic acid purification device according to the present embodiment may include a disk in which a fluid is transferred by a centrifugal force, a supply portion installed in the disk and configured to supply a specimen and a reagent required for nucleic acid purification, an adsorption reaction chamber which is installed in the disk and is connected with the supply portion and in which an adsorption medium for adsorbing a nucleic acid is accommodated and the nucleic acid is adsorbed from the specimen, and a solution accommodating portion which is connected with an output side of an adsorption reaction portion along a centrifugal direction of the disk and in which a solution discharged through the adsorption reaction chamber by the centrifugal force is accommodated and is discharged to the outside.

A nucleic acid purification device according to the present embodiment may include a disk in which a fluid is transferred by a centrifugal force, a supply portion installed in the disk to supply a specimen and a reagent, an adsorption reaction chamber which is installed in the disk and in which an adsorption medium is accommodated and a nucleic acid is adsorbed from the specimen supplied by the supply portion, a washing portion installed in the disk to wash the adsorption reaction chamber, a separation portion installed in the disk to elute the nucleic acid adsorbed to the adsorption medium, a solution accommodating portion which is installed in the disk and in which a solution discharged from the adsorption reaction chamber is separately accommodated, and a passage configured to control flow of the fluid moved according to the centrifugal force of the disk and a valve configured to selectively open/close the passage.

The supply portion, the adsorption reaction chamber, and the solution accommodating portion may be sequentially arranged at a rotational center of the disk along a centrifugal direction such that the specimen flows along the supply portion, the adsorption reaction chamber, and the solution accommodating portion by the centrifugal force.

The supply portion may include a reagent mixing chamber which is installed in the disk and in which a reagent for adsorbing the nucleic acid is accommodated and the specimen and the reagent are mixed with each other, a first passage which connects the reagent mixing chamber and the adsorption reaction chamber and through which a reagent mixture in a reagent mixing portion is transferred to the adsorption reaction chamber according to the centrifugal force of the disk, and a first valve configured to selectively open/close the first passage.

The supply portion may further include a separation accommodation portion installed in the disk and configured to separate a nontarget substance from the specimen.

The separation accommodation portion may include a separation chamber to which the specimen is supplied and in which the nontarget substance is separated by the centrifugal force of the disk, a second passage which connects the separation chamber and the reagent mixing chamber and through which a separated solution in the separation chamber is transferred to the reagent mixing chamber according to the centrifugal force of the disk, and a second valve configured to selectively open/close the second passage.

The supply portion may further include an enzyme supplying portion installed in the disk and connected with the separation chamber to supply a proteolytic enzyme.

The enzyme supplying portion may include an enzyme accommodating chamber which is installed in the disk and in which the proteolytic enzyme is accommodated, a third passage which connects the enzyme accommodating chamber and the separation chamber and through which the proteolytic enzyme is transferred to the separation chamber according to the centrifugal force of the disk, and a third valve configured to selectively open/close the third passage.

The supply portion may further include a reinforcing agent supplying portion installed in the disk and connected with the reagent mixing chamber to supply a reinforcing agent for reinforcing an attractive force between the nucleic acid and the adsorption medium.

The reinforcing agent supplying portion may include a reinforcing agent accommodating chamber which is installed in the disk and in which the reinforcing agent is accommodated, a fourth passage which connects the reinforcing agent accommodating chamber and the reagent mixing chamber and through which the reinforcing agent is transferred to the reagent mixing chamber according to the centrifugal force of the disk, and a fourth valve configured to selectively open/close the fourth passage.

The reinforcing agent may include isopropanol (IPA).

The nucleic acid purification device may further include a washing portion installed in the disk and connected with the adsorption reaction chamber to supply a washing liquid to the adsorption reaction chamber.

The washing portion may include a washing liquid accommodating chamber which is installed in the disk and in which a washing liquid is accommodated, a fifth passage which connects the washing liquid accommodating chamber and the adsorption reaction chamber and through which a washing liquid is transferred to the adsorption reaction chamber according to the centrifugal force of the disk, and a fifth valve configured to selectively open/close the fifth passage.

The nucleic acid purification device may further include a separation portion installed in the disk and connected with the adsorption reaction chamber to elute the nucleic acid adsorbed to the adsorption medium.

The separation portion may include an eluent accommodating chamber which is installed in the disk and in which an eluent is accommodated, a sixth passage which connects the eluent accommodating chamber and the adsorption reaction chamber and through which the eluent is transferred to the adsorption reaction chamber according to the centrifugal force of the disk, and a sixth valve configured to selectively open/close the sixth passage.

The solution accommodating portion may include a wasted solution accommodating chamber which is installed in the disk and is connected with the adsorption reaction chamber and in which a solution remaining in the adsorption reaction chamber after the nucleic acid is adsorbed to the adsorption medium is accommodated, a seventh passage which connects the adsorption reaction chamber and the wasted solution accommodating chamber and through which the solution is transferred to the wasted solution accommodating chamber according to the centrifugal force of the disk, a seventh valve configured to selectively open/close the seventh passage, a nucleic acid solution accommodating chamber which is connected with the adsorption reaction chamber and in which a nucleic acid elution solution separated from the adsorption medium is accommodated, an eighth passage which connects the adsorption reaction chamber and the nucleic acid solution accommodating chamber and through which the solution is transferred to the nucleic acid solution accommodating chamber according to the centrifugal force of the disk, and an eighth valve configured to selectively open/close the eighth passage.

The valve configured to open/close the passage of the disk may include a blocking member installed on the passage of the disk, formed of an elastic material, and configured to open/close the passage while elastically deformed, a pressing member disposed outside the blocking member and configured to selectively open/close the passage by pressing the blocking member by an external force, and a support installed in the disk and supporting the pressing member.

The nucleic acid purification device may further include a driver configured to selectively open/close the valve by applying an external force to the pressing member of the valve.

The adsorption medium may be at least one selected from the group consisting of a bead, a column, and a post on a silica surface and a bead on a chitosan surface.

The specimen may be biofluid including blood, lymphatic fluid, tissue fluid, and urine or cells or small cells including somatic cells, bacteria, and viruses.

The reagent may include a proteolytic enzyme for proteolysis, a reinforcing agent for reinforcing nucleic acid adsorption reaction for the adsorption medium, a washing liquid for washing the adsorption medium or an eluent for separating the nucleic acid adsorbed to adsorption medium.

The disk may be formed in a circular plate shape, and the plurality of adsorption reaction chambers may be arranged along a circumferential direction of the disk at intervals.

The nucleic acid purification device may further include a solution extraction port formed in the solution accommodating portion and configured to extract a solution from the disk to the outside as needed.

The nucleic acid purification device may further include a heating portion installed in the reagent mixing chamber and configured to heat an inner mixture.

The heating portion may include a heating element heated by an electromagnetic wave irradiated from the outside to apply thermal energy to the mixture.

The adsorption reaction chamber may include a gradient portion inclined such that a width thereof is narrowed toward an input side or an output side thereof.

A nucleic acid purification method according to the present embodiment may include a mounting step of mounting an adsorption medium for adsorbing a nucleic acid on an adsorption reaction chamber of a disk, an injecting step of injecting a specimen into the disk, an adsorbing step of adsorbing the nucleic acid by mixing the specimen with the adsorption medium by applying a centrifugal force to the adsorption reaction chamber, a removing step of removing a solution remaining in the adsorption reaction chamber after the nucleic acid is adsorbed by applying the centrifugal force to the adsorption reaction chamber, injecting an eluent into the adsorption reaction chamber; an eluting step of separating the nucleic acid from the adsorption medium by mixing the eluent with the adsorption medium by applying the centrifugal force to the adsorption reaction chamber, and a discharging step of discharging a solution in which the nucleic acid is eluted from the adsorption reaction chamber by applying the centrifugal force to the adsorption reaction chamber.

In the injecting step, a reagent for purifying the nucleic acid may be mixed with the specimen, and a mixture of the specimen and the reagent may be injected.

The mounting step includes mounting a reagent for purifying the nucleic acid on the disk.

The reagent may include a proteolytic enzyme for proteolysis, a reinforcing agent for reinforcing nucleic acid adsorption reaction for the adsorption medium, a washing liquid for washing the adsorption medium or an eluent for separating the nucleic acid adsorbed to the adsorption medium.

The nucleic acid purification method may further include a mixing step of mixing the specimen with a reagent for adsorbing the nucleic acid accommodated in a reagent mixing chamber, before the adsorbing step.

The mixing step may include opening a passage for transferring the specimen, transferring the specimen to the reagent mixing chamber by applying a centrifugal force by rotating the disk, closing the passage, and mixing the specimen with the reagent by rotating the disk through acceleration/deceleration.

In the mixing of the specimen with the reagent, a mixing time according to the rotation of the disk may be 5 minutes to 10 minutes.

The nucleic acid purification method may further include a separating step of separating a nontarget substance from a biomaterial, which is the specimen, by applying a centrifugal force to the specimen by rotating the disk, before the mixing step.

The nucleic acid purification method may further include a decomposing step of decomposing an unnecessary protein by mixing a proteolytic enzyme with the separated solution, after the separation step.

The decomposing step may include opening a passage for transferring the proteolytic enzyme, and transferring the proteolytic enzyme to the separated solution by applying a centrifugal force by rotating the disk.

The nucleic acid purification method may further include a reinforcing step of mixing a reinforcing agent for reinforcing an attractive force between the nucleic acid and the adsorption medium with a mixing liquid after the mixing step.

The reinforcing step may include opening a passage for transferring the reinforcing agent, transferring the reinforcing agent to the reagent mixing chamber by applying a centrifugal force by rotating the disk, and mixing the reinforcing agent by rotating the disk through acceleration/deceleration.

When the reinforcing agent is mixed, a mixing time according to the rotation of the disk may be 10 seconds to 60 seconds.

The reinforcing agent may include isopropanol (IPA).

The adsorbing step may include opening a passage for transferring a mixture in the reagent mixing chamber, transferring the mixture to the adsorption reaction chamber by applying a centrifugal force by rotating the disk, and mixing the mixture with the adsorption medium by rotating the disk through acceleration/deceleration.

When the mixture is mixed with the adsorption medium, a mixing time according to the rotation of the disk may be 1 minute to 5 minutes.

The removing step may include opening a passage for transferring a remaining solution after the adsorption is performed in the adsorption reaction chamber, and discharging the remaining solution to a wasted solution accommodating chamber by applying a centrifugal force by rotating the disk.

The removing step may further include a washing step of washing the adsorption medium by injecting a washing liquid into the adsorption reaction chamber.

The washing step may include opening a passage for transferring the washing liquid, transferring the washing liquid to the adsorption reaction chamber by applying a centrifugal force by rotating the disk, and discharging the washing liquid passing through the adsorption reaction chamber to the wasted solution accommodating chamber by applying a centrifugal force by rotating the disk.

The removing step may further include a drying step of drying the adsorption reaction chamber and the adsorption medium.

The drying step may include discharging the remaining solution to the wasted solution accommodating chamber by applying a centrifugal force to the adsorption reaction chamber by rotating the disk.

When the drying is performed, a drying time according to the rotation of the disk may be 1 minute to 3 minutes.

The eluting step may include closing a passage between the adsorption reaction chamber and the wasted solution accommodating chamber and opening a passage for transferring an eluent, transferring the eluent to the adsorption reaction chamber by applying a centrifugal force by rotating the disk, and mixing the eluent with the adsorption medium by rotating the disk through acceleration/deceleration.

When the eluent is mixed, a mixing time according to the rotation of the disk is 30 seconds to 2 minutes.

The discharging step may include opening a passage for transferring a nucleic acid elution solution in the adsorption reaction chamber, and discharging the nucleic acid elution solution to a nucleic acid solution accommodating chamber by applying a centrifugal force by rotating the disk.

### Advantageous Effects

In this way, according to the present embodiment, the entire processes of purifying a nucleic acid from various biofluids may be integrally performed. A time and an effort consumed for purifying the nucleic acid are minimized so that economical efficiency may be secured.

### DESCRIPTION OF THE DRAWINGS

FIG. 1 is a schematic view illustrating a nucleic acid purification device according to the present embodiment.

FIG. 2 is a schematic view for explaining an operation of the nucleic acid purification device according to the present embodiment.

FIG. 3 is a view illustrating the nucleic acid purification device in which components for purifying a cell-free nucleic acid are integrated according to the present embodiment.

FIG. 4 is a cross-sectional view schematically illustrating a valve configuration of the nucleic acid purification device according to the present embodiment.

FIG. 5 is a schematic view for explaining a nucleic acid purification process according to the present embodiment.

FIG. 6 is a graph depicting a result obtained by performing cell-free nucleic acid purification using the nucleic acid purification device according to the present embodiment.

FIG. 7 is a graph depicting a result obtained by performing cell-free nucleic acid purification and concentration using the nucleic acid purification device according to the present embodiment.

FIG. 8 is a graph depicting a result obtained by purifying a bacterial-derived nucleic acid using the nucleic acid purification device according to the present embodiment.

FIG. 9 is a graph depicting an experimental result according to driving conditions of the nucleic acid purification device according to the present embodiment.

### MODE FOR INVENTION

Hereinafter, embodiments of the present invention will be described with reference to the accompanying drawings such that those skilled in the art to which the present invention pertains may easily implement the present invention.

As easily understood by those skilled in the art to which the present invention pertains, the embodiments which will be described below may be modified in various forms without departing from the concept and scope of the present invention.

The same or similar components are designated by the same reference numerals as far as possible.

Terminologies used herein are merely for describing specific embodiments, and are not intended to limit the present invention.

A singular form used herein includes a plural form unless otherwise defined.

A term “include” used in the specification specifies specific characteristics, regions, essences, steps, operations, elements, and/or ingredients, and does not exclude existence or addition of other specific characteristics, regions, essences, steps, operations, elements, ingredients, and/or groups.

All terms including technical terms and scientific terms used herein have the same meanings as those generally understood by those skilled in the art to which the present invention pertains.

Terms defined in a dictionary are additionally interpreted to have meanings according with the related technical document and currently disclosed contents, and are not interpreted as ideally or very officially meanings unless otherwise defined.

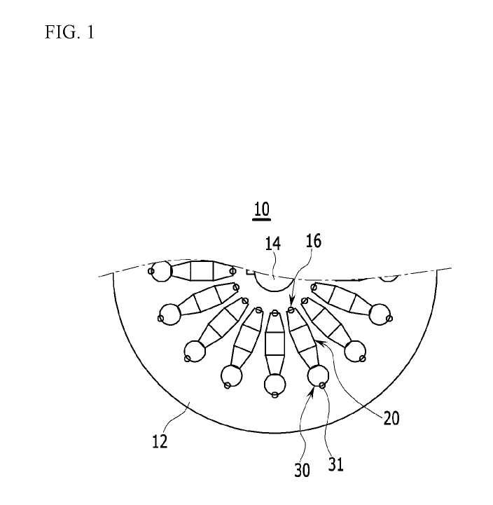

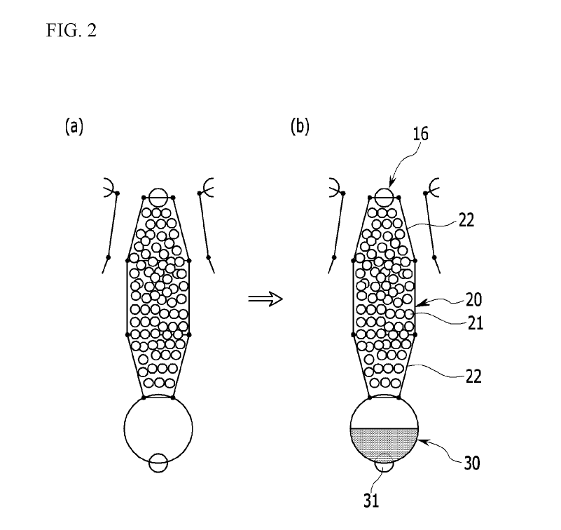

FIG. 1 is a schematic view for explaining a concept of the nucleic acid purification device according to the present embodiment, and FIG. 2 illustrates an operation of the nucleic acid purification device.

As illustrated in FIG. 1, a nucleic acid purification device 10 according to the present embodiment includes a disc 12 configured to generate a centrifugal force, supply portions 16 installed in the disk 12, adsorption reaction chambers 20, and solution accommodating portions 30.

The disk 12 may be formed in a circular plate structure.

A rotary shaft 14 is formed at the center of the disk 12, and the disk 12 is rotated about the rotary shaft 14 by a driving force provided from the outside.

A centrifugal force is generated according to the rotation of the disk 12, and the centrifugal force is applied to an internal fluid to feed the fluid.

The adsorption reaction chambers 20 are formed inside the disk 12.

The adsorption reaction chambers 20 may be understood as hollow spaces formed in the disk 12.

Adsorption mediums 21 configured to adsorb nucleic acids from specimens are accommodated inside the adsorption reaction chambers 20.

Accordingly, while the specimens pass through the adsorption reaction chambers 20, cell-free nucleic acids (DNAs) (hereinafter, referred to as nucleic acids) included in the specimens are adsorbed to the adsorption mediums 21.

In the present embodiment, the specimens may be biofluid including blood, lymphatic fluid, tissue fluid, and urine or cells or small cells including somatic cells, bacteria, and viruses.

Also, the adsorption mediums 21 may be at least one selected from the group consisting of a bead, a column, and a post on a silica surface and a bead on a chitosan surface.

The plurality of adsorption reaction chambers 20 may be arranged and installed at intervals along a circumferential direction of the disk 12.

Also, since the specimens are moved in a centrifugal direction by the centrifugal force generated when the disk 12 is rotated, the adsorption reaction chambers 20 may be elongated from the center toward an outer tip end of the disk 12 along the centrifugal direction.

Also, the adsorption reaction chambers 20 may have gradient portions 22 formed at input sides or output sides thereof and inclined with respect to the centrifugal direction, that is, in a direction in which the specimens flow.

As illustrated in FIG. 1, the gradient portions 22 may have inner surfaces inclined such that the widths thereof are narrowed toward the input sides and/or the output sides of the adsorption reaction chambers 20.

In this way, the gradient portions 22 may be formed in the adsorption reaction chambers 20, thereby providing smooth flow of solutions inside the adsorption reaction chambers 20.

The support portions 16 are arranged on the input sides of the adsorption reaction chambers 20, facing a rotational center of the disk 12, along the centrifugal direction of the disk 12, and are connected with the adsorption reaction chambers 20.

The solution accommodating portions 30 are arranged on the output sides of the adsorption reaction chambers 20, corresponding to an opposite side and facing the outer tip end of the disk 12, and are connected with the adsorption reaction chambers 20.

In the following description, the input sides mean sides of the corresponding chambers, into which a fluid is introduced and which are close to the rotational center of the disk 12 along the centrifugal direction, and the output sides mean sides of the corresponding chambers, from which the fluid is discharged and which are close to the outer tip end of the disk 12 along the centrifugal direction.

In this way, the supply portions 16, the adsorption reaction chambers 20, and the solution accommodating portions 30 are sequentially arranged at the rotational center of the disk 12 along the centrifugal direction, so that the specimens supplied through the supply portions 16 may sequentially flow to the solution accommodating portions 30 via the adsorption reaction chambers 20 by the centrifugal force generated when the disk 12 is rotated.

The supply portions 16 supply the specimens and reagents required for purifying the nucleic acids into the adsorption reaction chambers 20.

The supply portions 16, which have passage structures communicating with the adsorption reaction chambers 20, may have structures configured to separately supply the specimens and the reagents from the outside of the disk 12 through tip ends of the input sides of the adsorption reaction chambers 20.

In addition to the above-described structures, the supply portions 16 may be separately installed inside the disk 12.

In the case of these structures, the entire processes from supplying of the specimens to purifying of the nucleic acids may be integrated and may be collectively performed.

Description thereof will be made again later.

The reagents, which are materials for adsorbing the nucleic acids to the adsorption mediums 21, may be supplied to the adsorption reaction chambers 20 through the supply portions 16.

Also, the reagents may include proteolytic enzymes for proteolysis, reinforcing agents for reinforcing nucleic acid adsorption reaction for adsorption mediums, washing solutions for washing adsorption mediums or eluents for separating nucleic acids adsorbed to adsorption mediums.

In the present embodiment, the specimens may be supplied to the adsorption reaction chambers 20 through the supply portions 16 separately or while being mixed with the reagents.

The solution accommodating portions 30 may be understood as hollow spaces formed in the disk 12, and are connected with the output sides of the adsorption reaction chambers 20.

The solution accommodating portions 30 may have solution extraction ports 31 through which solutions transferred into the solution accommodating portions 30 are to be discharged to the outside of the disk 12.

Accordingly, if necessary, the solutions transferred to the solution accommodating portions 30 may be discharged to the outside through the solution extraction ports 31.

Hereinafter, a nucleic acid purification operation through the nucleic acid purification device will be described with reference to FIG. 2.

First, the adsorption mediums 21 are mounted and prepared inside the adsorption reaction chambers 20 of the disk 12.

When the preparation is completed, solutions obtained by mixing specimens with reagents for nucleic acid purification are supplied into the disk 12 through the supply portions 16.

The solutions supplied through the supply portions 16 may be further mixed with proteolytic enzymes and/or reinforcing agents for reinforcing nucleic acid adsorption reaction as the reagents.

A mixed liquid of the specimens and the reagents supplied into the disk 12 is introduced into the input sides of the adsorption reaction chambers 20.

In this state, the disk 12 is rotated to apply a centrifugal force to the adsorption reaction chambers 20.

By the centrifugal force of the disk 12, the specimen mixed liquid is mixed with the adsorption mediums 21 accommodated inside the adsorption reaction chambers 20 while flowing from the input sides to the output sides of the adsorption reaction chambers 20.

In this process, nucleic acids included in the mixed liquid are adsorbed to the adsorption mediums 21.

The mixed liquid flowing along the adsorption reaction chambers 20 is discharged to the solution accommodating portions 30 connected with the output sides of the adsorption reaction chambers 20 by the centrifugal force of the disk 12.

The specimen mixed liquid passing through the adsorption reaction chambers 20 is a residual solution obtained by separating the nucleic acids while the nucleic acids are adsorbed to the adsorption mediums 21, and the residual solution is discharged to the solution accommodating portions 30 and is removed in the adsorption reaction chambers 20.

The residual solution discharged to the solution accommodating portions 30 is discharged to the outside of the disk 12 through the solution extraction ports 31 of the solution accommodating portions 30.

When the residual solution obtained by separating and adsorbing the nucleic acids is completely removed in the adsorption reaction chambers 20, the nucleic acids are separated and extracted from the adsorption mediums 21 inside the adsorption reaction chambers 20.

In the present embodiment, before the nucleic acids are separated and extracted, a washing operation for the adsorption reaction chambers 20 and the adsorption mediums 21 may be performed.

For the washing operation, a washing liquid is injected through the supply portions 16 and is supplied to the adsorption reaction chambers 20.

In this state, the disk 12 is rotated to apply the centrifugal force to the adsorption reaction chambers 20.

The washing liquid washes the insides of the adsorption reaction chambers 20 and the surfaces of the accommodated adsorption mediums 21 while flowing from the input sides to the output sides of the adsorption reaction chambers 20 by the centrifugal force of the disk 12.

The washing liquid is discharged to the solution accommodating portions 30 connected with the output sides of the adsorption reaction chambers 20 by the centrifugal force of the disk 12.

The washing liquid discharged to the solution accommodating portions 30 is discharged to the outside of the disk 12 through the solution extraction ports 31 of the solution accommodating portions 30.

Also, in the present embodiment, after the washing operation, a process for drying the adsorption mediums 21 may be further performed.

In order to dry the adsorption mediums 21 to which the nucleic acids are adsorbed, after the washing operation, the disk 12 is rotated to apply a centrifugal force to adsorption reacting portions.

Accordingly, even after the washing, both the washing liquid and the residual solution remaining in the adsorption reaction chambers 20 and the surfaces of the adsorption mediums 21 are discharged and removed to the solution accommodating portions 30.

The solution discharged to the solution accommodating portions 30 is discharged to the outside of the disk 12 through the solution extraction ports 31.

After the above operation, the nucleic acids are separated and extracted from the adsorption mediums 21 inside the adsorption reaction chambers 20.

Finally, the eluents are injected through the supply portions 16.

The eluents supplied into the disk 12 through the supply portions 16 are introduced into the input sides of the adsorption reaction chambers 20.

In this state, the disk 12 is rotated to apply a centrifugal force to the adsorption reaction chambers 20.

The eluents are mixed with the adsorption mediums 21 while flowing from the input sides to the output sides of the adsorption reaction chambers 20 by the centrifugal force of the disk 12, and in this process, the nucleic acids adsorbed to the adsorption mediums 21 are separated from the adsorption mediums 21 by the eluents.

When the nucleic acids are separated from the adsorption mediums 21, the disk 12 is rotated so that solutions in which the nucleic acids are eluted are discharged to the solution accommodating portions 30.

The solutions in which the nucleic acids are eluted are discharged to the solution accommodating portions 30 connected with the output sides of the adsorption reaction chambers 20 by the centrifugal force of the disk 12.

In this way, since the nucleic acids may be easily purified from the specimens and may be extracted to the solution accommodating portions 30, the nucleic acids may be utilized for a necessary operation such as molecular diagnosis.

Hereinafter, in yet another embodiment, an automated nucleic acid purification device which may integrally perform the entire nucleic acid purification processes will be described.

Hereinafter, the above-described configurations are designated by the same reference numerals, and detail description thereof will be omitted.

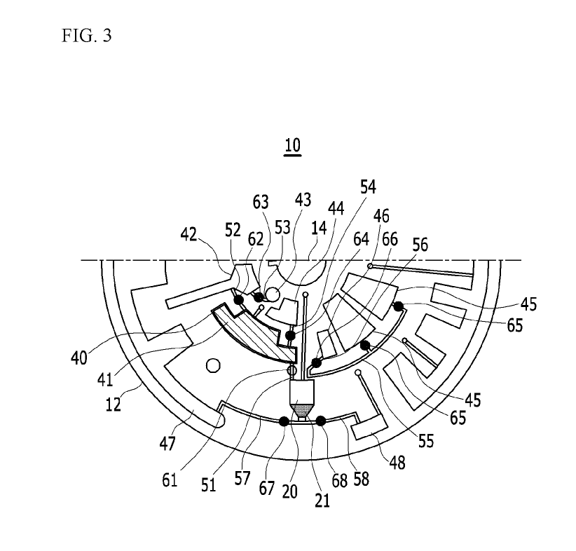

FIG. 3 illustrates the nucleic acid purification device in which configurations for nucleic acid purification are integrated according to the present embodiment.

As illustrated in FIG. 3, the nucleic acid purification device according to the present embodiment includes a disk 12 configured to transfer a fluid by a centrifugal force, a supply portion installed in the disk 12 to supply a specimen and a reagent, an adsorption reaction chamber 20 in which an adsorption medium 21 is accommodated and a nucleic acid is adsorbed, a washing portion configured to wash the adsorption reaction chamber 20, a separation portion configured to elute the nucleic acid adsorbed to the adsorption medium 21, a solution accommodating portion configured to separate and accommodate a solution discharged from the adsorption reaction chamber 20, a passage configured to control flow of the fluid moved according to the centrifugal force of the disk 12, and a valve configured to selectively open/close the passage.

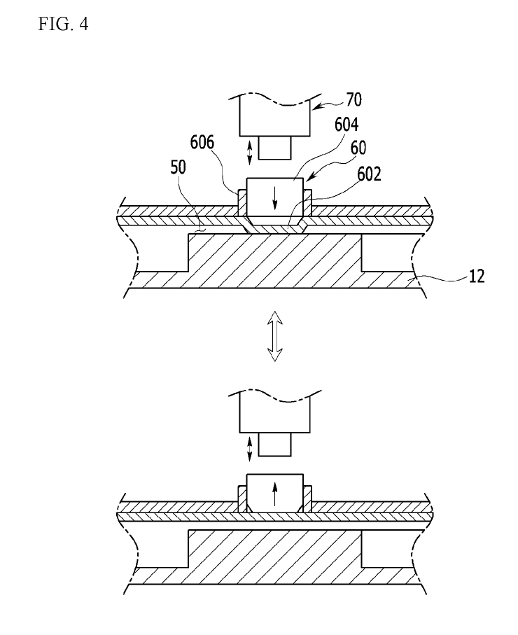

Also, the present device further includes a driver 70 (see FIG. 4) configured to drive the valve to open/close the passage.

Accordingly, when the driver operates the valve by applying an external force to the valve, the flow of the fluid is controlled as the passage is opened or closed. The specimen is transferred from the supply portion to the adsorption reaction chamber 20 and the solution accommodating portion by the centrifugal force of the disk 12, and is then purified. The driver may be variously modified as long as the drive may apply an external force to the valve.

The supply portion may include a reagent mixing chamber 40 installed in the disk 12, having the reagent for nucleic acid adsorption accommodated therein, and configured to mix the specimen and the reagent with each other.

The reagent mixing chamber 40 may be understood as a hollow space formed in the disk 12.

An inlet through which the reagent is injected may be formed on one side of the reagent mixing chamber 40.

The reagent mixing chamber 40 and the adsorption reaction chamber 20 are sequentially arranged from the rotational center toward an outer tip end of the disk 12 along a centrifugal direction of the disk 12, so that the fluid flows from the reagent mixing chamber 40 to the adsorption reaction chamber 20.

The reagent mixing chamber 40 is connected with an input side of the adsorption reaction chamber 20.

A necessary reagent may be accommodated in the reagent mixing chamber 40 through the inlet in advance.

Accordingly, the specimen introduced into the reagent mixing chamber 40 is mixed with the reagent accommodated in the reagent mixing chamber 40. Thereafter, the nucleic acid is adsorbed in the adsorption reaction chamber 20.

In the present embodiment, the reagent mixing chamber 40 further includes a heating portion configured to heat an inner solution.

The heating portion may include a heating element 41 installed in the reagent mixing chamber 40 and heated by an electromagnetic wave irradiated from the outside, to apply thermal energy to the solution.

Accordingly, when the electromagnetic wave is irradiated from the outside of the disk 12 as needed, the heating element 41 installed in the reagent mixing chamber 40 is heated by the electromagnetic wave to apply the thermal energy to the inner solution.

Thus, the solution inside the reagent mixing chamber 40 is heated by the heating element 41 so that the temperature of the solution may increase.

The temperature of the solution in the reagent mixing chamber 40 increases, so that nucleic acid adsorption reaction efficiency may be further improved, a nucleic acid purification time may be reduced, and a driving apparatus of the nucleic acid purification device may be further simplified.

The passage (hereinafter, for convenience of description, referred to as a first passage 51) through which a reagent mixture mixed inside a reagent mixing portion is transferred to the adsorption reaction chamber 20 according to the centrifugal force of the disk 12 is formed between the reagent mixing chamber 40 and the adsorption reaction chamber 20.

The valve (hereinafter, for convenience of description, referred to as a first valve 61) configured to selectively open/close the first passage 51 is installed on the first passage 51.

Accordingly, when the valve is driven, the flow of the fluid through the passage may be controlled as the passage formed in the disk 12 is opened or closed.

The structures of the passage and the valve will be described below.

The first passage 51 connects an output side of the reagent mixing chamber 40 and an input side of the adsorption reaction chamber 20, and allows the fluid to flow from the reagent mixing chamber 40 to the adsorption reaction chamber 20 according to the centrifugal force of the disk 12.

Accordingly, in a state in which the first valve 61 is operated so that the first passage 51 is opened, when the centrifugal force is applied as the disk 12 is rotated, the solution inside the reagent mixing chamber 40 flows from the reagent mixing chamber 40 through the first passage 51 to the adsorption reaction chamber 20.

The adsorption reaction chamber 20 has the adsorption medium 21 accommodated therein, is arranged between the reagent mixing chamber 40 and the solution accommodating portion, and has the input side connected to the reagent mixing chamber 40 through the first passage 51 and the output side connected to the solution accommodating portion through a separate passage.

Also, in the present embodiment, the supply portion may further include a separation accommodation portion installed in the disk 12 to separate a nontarget substance from the specimen supplied to the reagent mixing chamber 40, an enzyme supplying portion installed in the disk 12 to supply proteolytic enzymes to the separation accommodation portion, or a reinforcing agent supplying portion installed in the disk 12 and connected with the reagent mixing chamber 40 to supply a reinforcing agent for reinforcing an attractive force between the nucleic acid and the adsorption medium 21.

The separation accommodating portion includes a separation chamber 42 in which the nontarget substance is separated by the centrifugal force of the disk 12, a second passage 52 which connects the separation chamber 42 and the reagent mixing chamber 40 and through which a separated solution inside the separation chamber 42 is transferred to the reagent mixing chamber 40 according to the centrifugal force of the disk 12, and a second valve 62 configured to selectively open/close the second passage 52.

The nontarget substance means a substance other than a target substance to be purified.

In the present embodiment, the nontarget substance may mean substances other than the cell-free nucleic acid.

The specimen supplied to the separation chamber 42 is centrifuged and purified by the centrifugal force according to the rotation of the disk 12.

Accordingly, the specimen is separated into a solution including a nucleic acid and a solid matter, the solid matter is pushed toward the outer tip end of the disk 12 along the centrifugal direction, and the solution separated from the solid matter is located toward the center of the disk 12.

For example, when the specimen is blood, the blood is separated into blood cells corresponding to the solid matter and blood plasma corresponding to a liquid component by the centrifugation.

The separation chamber 42 may be understood as a hollow space formed in the disk 12.

An inlet through which the specimen is to be injected may be formed on one side of the separation chamber 42.

The separation chamber 42 is connected to an input side of the reagent mixing chamber 40 through the second passage 52.

As illustrated in FIG. 3, the separation chamber 42 may be formed to have a tubular shape extending along the centrifugal direction such that the solution is clearly separated in a space.

The second passage 52 may be connected with a boundary point between the solid matter and the solution separated in the separation chamber 42.

Accordingly, the solid matter separated in the separation chamber 42 may continuously remain in the separation chamber 42, and only the solution including the nucleic acid may be transferred to the reagent mixing chamber 40 through the second passage 52.

The second passage 52 allows the fluid to flow from the separation chamber 42 to the reagent mixing chamber 40 according to the centrifugal force of the disk 12.

The second valve 62 configured to selectively open/close the second passage 52 is installed on the second passage 52.

Accordingly, when the second valve 62 is driven, the flow of the fluid through the second passage 52 may be controlled as the second passage 52 formed in the disk 12 is opened or closed.

Thus, in a state in which the second valve 62 is operated so that the second passage 52 is opened, when the centrifugal force is applied according to the rotation of the disk 12, the solution separated in the separation chamber 42 flows to the reagent mixing chamber 40 through the second passage 52.

The enzyme supplying portion includes an enzyme accommodating chamber 43 installed in the disk 12 to accommodate the proteolytic enzymes, a third passage 53 which connects the enzyme accommodating chamber 43 and the separation chamber 42 and through which the proteolytic enzymes are transferred to the separation chamber 42 according to the centrifugal force of the disk 12, and a third valve 63 configured to selectively open/close the third passage 53.

In the present embodiment, the proteolytic enzymes are mixed with the solution separated from the specimen to decompose unnecessary proteins other than the nucleic acid.

The enzyme accommodating chamber 43 may be understood as a hollow space formed in the disk 12.

An inlet through which the proteolytic enzymes are to be injected may be formed on one side of the enzyme accommodating chamber 43.

The proteolytic enzymes may be accommodated in the enzyme accommodating chamber 43 through the inlet in advance.

The enzyme accommodating chamber 43 may be connected to the separation chamber 42 through the third passage 53.

The third passage 53 is connected with an output side of the enzyme accommodating chamber 43 and is connected with an elongated space in which the solution separated in the separation chamber 42 is accommodated, that is, a tip end on the rotational center of the disk 12.

Accordingly, the proteolytic enzymes accommodated in the enzyme accommodating chamber 43 may flow to a solution accommodating space of the separation chamber 42 along the third passage 53 by the centrifugal force of the disk 12.

The proteolytic enzymes supplied to the separation chamber 42 are mixed with the solution inside the separation chamber 42 to decompose unnecessary proteins in the solution.

The third valve 63 configured to selectively open/close the third passage 53 is installed on the third passage 53.

Accordingly, when the third valve 63 is driven, movement of the proteolytic enzymes through the third passage 53 may be controlled as the third passage 53 formed in the disk 12 is opened or closed.

In the present embodiment, it is sufficient for the proteolytic enzymes to move to the separation chamber 42 along the third passage 53 by the centrifugal force, and a formation position of the enzyme accommodating chamber 43 with respect to the disk 12 is not particularly limited.

The reinforcing agent supplying portion includes a reinforcing agent accommodating chamber 44 installed in the disk 12 and accommodating the reinforcing agent, a fourth passage 54 which connects the reinforcing agent accommodating chamber 44 and the reagent mixing chamber 40 and though which the reinforcing agent is transferred to the reagent mixing chamber 40 according to the centrifugal force of the disk 12, and a fourth valve 64 configured to selectively open/close the fourth passage 54.

In the present embodiment, the reinforcing agent may include isopropanol (IPA).

The reinforcing agent is transferred to the reagent mixing chamber 40 and is mixed with the solution mixed with the reagent to reinforce the attractive force between the nucleic acid and an adsorption surface of the adsorption medium 21.

The reinforcing agent accommodating chamber 44 may be understood as a hollow space formed in the disk 12.

An inlet through which a reinforcing agent is to be injected may be formed on one side of the reinforcing agent accommodating chamber 44.

The reinforcing agent may be accommodated in the reinforcing agent accommodating chamber 44 through the inlet in advance.

The reinforcing agent accommodating chamber 44 is connected to the reagent mixing chamber 40 through the fourth passage 54.

The fourth passage 54 connects an output side of the reinforcing agent accommodating chamber 44 and the input side of the reagent mixing chamber 40, and thus enables the reinforcing agent accommodated in the reinforcing agent accommodating chamber 44 to flow to the reagent mixing chamber 40 along the fourth passage 54 by the centrifugal force of the disk 12.

The fourth valve 64 configured to selectively open/close the fourth passage 54 is installed on the fourth passage 54.

Accordingly, when the fourth valve 64 is driven, movement of the reinforcing agent through the fourth passage 54 may be controlled as the fourth passage 54 formed in the disk 12 is opened or closed.

In the present embodiment, it is sufficient for the reinforcing agent to move to the reagent mixing chamber 40 along the fourth passage 54, and a formation position of the reinforcing agent accommodating chamber 44 with respect to the disk 12 is not particularly limited.

The washing portion includes, which is configured to wash the adsorption medium 21 and an inside of the adsorption reaction chamber 20 after the nucleic acid is adsorbed to the adsorption medium 21 in the adsorption reaction chamber 20, washing liquid accommodating chambers 45 which is installed in the disk 12 and in which a washing liquid is accommodated, a fifth passage 55 which connects the washing liquid accommodating chambers 45 and the adsorption reaction chamber 20 and through which the washing liquid is transferred to the adsorption reaction chamber 20 according to the centrifugal force of the disk 12, and fifth valves 65 configured to selectively open/close the fifth passage 55.

The washing liquid washes and removes the reagent remaining in an inner surface of the adsorption reaction chamber 20 or the surface of the adsorption medium 21.

The washing liquid accommodating chambers 45 may be understood as hollow spaces formed in the disk 12.

Inlets through which the washing liquid is to be injected may be formed on sides of the washing liquid accommodating chambers 45.

The washing liquid may be accommodated in the washing liquid accommodating chambers 45 through the inlets in advance.

In the present embodiment, the washing liquid accommodating chambers 45 may be provided in plurality.

Accordingly, the inside of the adsorption reaction chamber 20 may be washed several times.

The washing liquid accommodating chambers 45 are connected with the adsorption reaction chamber 20 through the fifth passage 55.

The fifth passage 55 connects output sides of the washing liquid accommodating chambers 45 and the input side of the adsorption reaction chamber 20.

Accordingly, the washing liquid accommodated in the washing liquid accommodating chambers 45 may flow into the adsorption reaction chamber 20 along the fifth passage 55 by the centrifugal force of the disk 12.

After washing the inside of the adsorption reaction chamber 20, the washing liquid supplied to the adsorption reaction chamber 20 is discharged to the solution accommodating portion through a passage connected with the output side of the adsorption reaction chamber 20.

The fifth valves 65 configured to selectively open/close the fifth passage 55 are installed on the fifth passage 55.

Accordingly, when the fifth valves 65 are driven, flow of the washing liquid through the fifth passage 55 may be controlled as the fifth passage 55 formed in the disk 12 is opened or closed.

Also, as illustrated in FIG. 3, when the washing liquid accommodating chambers 45 are provided in plurality, a separate passage and a valve configured to open/close the passage may be installed between each of the washing liquid accommodating chambers 45 and the fifth passage 55.

In the present embodiment, it is sufficient for the washing liquid to move to the adsorption reaction chamber 20 along the fifth passage 55 by the centrifugal force, and formation positions of the washing liquid accommodating chambers 45 with respect to the disk 12 are not particularly limited.

The separation portion includes an eluent accommodating chamber 46 which is installed in the disk 12 and in which an eluent is accommodated, a sixth passage 56 which connects the eluent accommodating chamber 46 and the adsorption reaction chamber 20 and through which the eluent is transferred to the adsorption reaction chamber 20 according to the centrifugal force of the disk 12, and a sixth valve 66 configured to selectively open/close the sixth passage 56.

The eluent elutes the nucleic acid adsorbed to the adsorption medium 21 and separates the nucleic acid from the adsorption medium 21.

The eluent accommodating chamber 46 may be understood as a hollow space formed in the disk 12.

An inlet through which the eluent is to be injected may be formed on one side of the eluent accommodating chamber 46.

The eluent may be accommodated in the eluent accommodating chamber 46 through the inlet in advance.

In the present embodiment, the eluent accommodating chamber 46 is connected to the adsorption reaction chamber 20 through the sixth passage 56.

The sixth passage 56 connects an output side of the eluent accommodating chamber 46 and the input side of the adsorption reaction chamber 20.

Accordingly, the eluent accommodated in the eluent accommodating chamber 46 may flow into the adsorption reaction chamber 20 along the sixth passage 56 by the centrifugal force of the disk 12.

The eluent supplied to the adsorption reaction chamber 20 is mixed with the adsorption medium 21 inside the adsorption reaction chamber 20, and in this process, the nucleic acid adsorbed to the adsorption medium 21 is eluted and separated from the adsorption medium 21.

The nucleic acid separated from the adsorption medium 21 together with the eluent is discharged to the solution accommodating portion through a passage connected with the output side of the adsorption reaction chamber 20.

The sixth valve 66 configured to selectively open/close the sixth passage 56 is installed on the sixth passage 56.

Accordingly, when the sixth valve 66 is driven, flow of the eluent through the sixth passage 56 may be controlled as the sixth passage 56 formed in the disk 12 is opened or closed.

In the present embodiment, it is sufficient for the eluent to move to the adsorption reaction chamber 20 along the sixth passage 56 by the centrifugal force, and a formation position of the eluent accommodating chamber 46 with respect to the disk 12 is not particularly limited

The solution accommodating portion includes a wasted solution accommodating chamber 47 which is installed in the disk 12 and is connected to the adsorption reaction chamber 20 and in which the residual solution remaining in the adsorption reaction chamber 20 after the nucleic acid is adsorbed to the adsorption medium 21 is accommodated, a seventh passage 57 which connects the adsorption reaction chamber 20 and the wasted solution accommodating chamber 47 and through which the solution is transferred to the wasted solution accommodating chamber 47 according to the centrifugal force of the disk 12, a seventh valve 67 configured to selectively open/close the seventh passage 57, a nucleic acid solution accommodating chamber 48 which is connected to the adsorption reaction chamber 20 and in which a nucleic acid elution solution separated from the adsorption medium 21 is accommodated, an eighth passage 58 which connects the adsorption reaction chamber 20 and the nucleic acid solution accommodating chamber 48 and through which the solution is transferred to the nucleic acid solution accommodating chamber 48 according to the centrifugal force of the disk 12, and an eight valve 68 configured to selectively open/close the eighth passage 58.

The solution accommodating portion includes two chambers which are divided into the nucleic acid solution accommodating chamber 48 in which the solution including the purified nucleic acid is accommodated and the wasted solution accommodating chamber 47 in which the to-be-wasted solution is accommodated.

Accordingly, finally, the purified nucleic acid may be separately separated and extracted through the nucleic acid solution accommodating chamber 48.

The to-be-wasted solution means a solution not including the nucleic acid corresponding to the target substance, such as the residual solution after the nucleic acid is adsorbed to the adsorption medium 21 and the washing liquid, and the nucleic acid solution means a solution eluted by the eluent and including the nucleic acid corresponding to the target substance.

The wasted solution accommodating chamber 47 and the nucleic acid solution accommodating chamber 48 may be separated from each other and may be understood as hollow spaces formed in the disk 12.

The to-be-wasted solution such as the residual solution in the adsorption reaction chamber 20 and the washing liquid is accommodated in the wasted solution accommodating chamber 47.

The wasted solution accommodating chamber 47 is connected to the adsorption reaction chamber 20 through the seventh passage 57.

The seventh passage 57 connects the output side of the adsorption reaction chamber 20 and an input side of the wasted solution accommodating chamber 47.

Accordingly, the residual solution or the washing liquid discharged from the adsorption reaction chamber 20 is discharged to the wasted solution accommodating chamber 47 along the seventh passage 57 by the centrifugal force of the disk 12.

The seventh valve 67 configured to selectively open/close the seventh passage 57 is installed on the seventh passage 57.

Accordingly, when the seventh valve 67 is driven, flow of the solution discharged through the seventh passage 57 may be controlled as the seventh passage 57 formed in the disk 12 is opened or closed.

In the present embodiment, it is sufficient for the solution to move to the wasted solution accommodating chamber 47 along the seventh passage 57 by the centrifugal force, and a formation position of the wasted solution accommodating chamber 47 with respect to the disk 12 is not particularly limited.

The solution including the nucleic acid separated from the adsorption medium 21 of the adsorption reaction chamber 20 is accommodated in the nucleic acid solution accommodating chamber 48.

The nucleic acid solution accommodating chamber 48 is connected to the adsorption reaction chamber 20 through the eighth passage 58.

The eighth passage 58 connects the output side of the adsorption reaction chamber 20 and an input side of the nucleic acid solution accommodating chamber 48.

Accordingly, the solution including the nucleic acid discharged from the adsorption reaction chamber 20 is discharged to the nucleic acid solution accommodating chamber 48 along the eighth passage 58 by the centrifugal force of the disk 12.

The eighth valve 68 configured to selectively open/close the eighth passage 58 is installed on the eighth passage 58.

Accordingly, when the eighth valve 68 is driven, flow of the solution discharged through the eighth passage 58 may be controlled as the eighth passage 58 is opened or closed.

In the present embodiment, it is sufficient for the solution to move to the nucleic acid solution accommodating chamber 48 along the eighth passage 58 by the centrifugal force, and a formation position of the nucleic acid solution accommodating chamber 48 with respect to the disk 12 is not particularly limited.

FIG. 4 illustrates a structure of a passage and a valve formed in the disk 12 according to the present embodiment.

In the following description, the first passage 51 to the eighth passage 58 have different sizes, different lengths, and different formation positions, but are the same in that the first passage 51 to the eighth passage 58 serve as a conduit for transferring a fluid.

Accordingly, the first passage 51 to the eighth passage 58 will be referred to as a passage 50 below.

The first valve 61 to the eighth valve 68 have the same structure, and will be referred to as a valve 60 below.

As illustrated in FIG. 4, the valve configured to open/close the passage formed in the disk 12 may include a blocking member 602 installed on the passage of the disk 12, formed of an elastic material, and configured to open/close the passage while being elastically deformed, a pressing member 604 disposed outside the blocking member 602 and configured to selectively open/close the passage by pressing the blocking member 602 by an external force, and a support 606 installed in the disk 12 and supporting the pressing member 604.

The support 606 may support the pressing member 604, and may fix a state in which the pressing member 604 is pushed by the external force or may fix a state in which the pressing member 604 returns to an original position by the external force.

Accordingly, for example, when the pressing member 604 is pressed by the external force, the pressing member 604 is moved to press the blocking member 602.

Thus, the blocking member 602 is elastically deformed to block the passage so as to block flow of the fluid.

When the pressing member 604 is moved in an opposite direction by the external force, the pressure by the pressing member 604 is released so that the blocking member 602 returns to an original state by an elastic force thereof.

Accordingly, the passage blocked by the blocking member 602 is opened so that the fluid may flow.

The valve may use a push switch scheme in which when an external force is applied to the pressing member 604, the pressing member 604 is pushed, and when an external force is applied to the pressing member 604 again, the pressing member 604 returns to an original position.

The operation scheme of the valve may be variously changed, and all structures may be applied in which the pressing member 604 is pushed by an external force to press the blocking member 602 or the pressing member 604 returns to an original position so that the pressure to the blocking member 602 is released.

The valve is opened/closed according to an operation of the driver 70 disposed outside the disk 12.

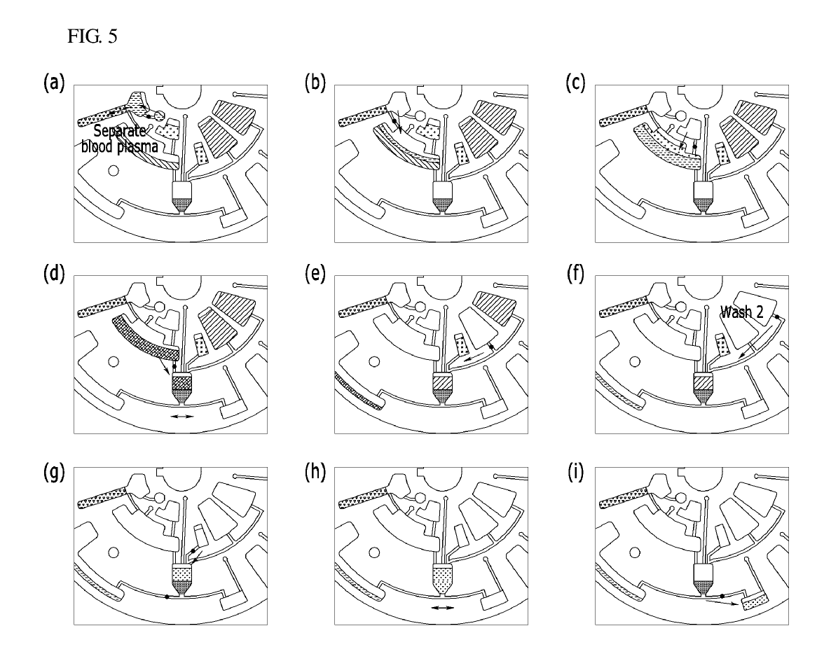

The driver 70 moves to a position of a valve of each passage according to the operation process, and drives the valve of the corresponding passage. Hereinafter, a nucleic acid purification operation through the nucleic acid purification device will be described with reference to FIGS. 3 and 5.

First, a reagent necessary for nucleic acid purification is mounted and prepared on the disk 12.

As the reagent, the adsorption medium 21, the proteolytic enzymes, the reagent, the reinforcing agent, the washing liquid, and the eluent may be mounted on the adsorption reaction chamber 40, the enzyme accommodating chamber 43, the reagent mixing chamber 40, the reinforcing agent accommodating chamber 44, the washing liquid accommodating chambers 45, and the eluent accommodating chamber 46, respectively.

In a preparation state, the valve installed in the passage of the disk 12 is closed so that the passage is maintained closed.

When preparation is completed, the specimen is injected into the separation chamber 42 of the disk 12.

When the specimen is injected into the separation chamber 42, the disk 12 is rotated to apply a centrifugal force to the separation chamber 42.

The specimen injected into the separation chamber 42 is centrifuged by the centrifugal force of the disk 12.

When the specimen is completely centrifuged, the third valve 63 of the disk 12 is opened through the driver 70 (see FIG. 4) so that the third passage 53 is opened.

The third passage 53 is opened, and the disk 12 is rotated to apply the centrifugal force to the enzyme accommodating chamber 43.

The proteolytic enzymes accommodated in the enzyme accommodating chamber 43 flow through the third passage 53, are transferred to the separation chamber 42, and are mixed by the centrifugal force of the disk 12 (see FIG. 5A).

Next, the second valve 62 is opened by the driver so that the second passage 52 is opened.

The second passage 52 is opened, and the disk 12 is rotated to apply a centrifugal force to the separation chamber 42.

A mixed liquid of the separated solution and the proteolytic enzymes in the separation chamber 42 flows through the second passage 52 and is transferred to the reagent mixing chamber 40 by the centrifugal force of the disk 12.

When all the mixed liquid is transferred to the reagent mixing chamber 40, the second valve 62 is driven to close the second passage 52.

Further, the disk 12 is repeatedly rotated forward/rearward through acceleration/deceleration so that the mixed liquid and the reagent are mixed with each other in the reagent mixing chamber 40 (see FIG. 5B).

In the present embodiment, a mixing time during which the specimen and the reagent are mixed with each other in the reagent mixing chamber 40 may be 5 minutes to 10 minutes.

When the mixing time is smaller than 5 minutes, since the mixing is not properly performed, adsorption efficiency of the nucleic acid deteriorates, and when the mixing time is larger than 10 minutes, only a purification time becomes longer, there is no increase in an effect, and the specimen may be damaged.

When the reagent is completely mixed, the fourth valve 64 is operated by the driver to open the fourth passage 54.

The fourth passage 54 is opened, and the disk 12 is rotated to apply a centrifugal force to the reinforcing agent accommodating chamber 44.

The reinforcing agent in the reinforcing agent accommodating chamber 44 flows through the fourth passage 54 and is transferred to the reagent mixing chamber 40 by the centrifugal force of the disk 12.

The disk 12 is rotated forward/rearward through acceleration/deceleration so that the reinforcing agent is mixed with the solution in the reagent mixing chamber 40 (see FIG. 5C).

In the present embodiment, a mixing time during which the reinforcing agent is mixed in the reagent mixing chamber 40 may be 10 seconds to 60 seconds.

When the mixing time is smaller than 10 seconds, since the mixing is not properly performed, adsorption efficiency of the nucleic acid deteriorates, and when the mixing time is larger than 60 seconds, there is no increase in an effect, and the specimen may be damaged.

When all the mixing reaction is completed, the first valve 61 is operated by the driver to open the first passage 51.

The first passage 51 is opened, and the disk 12 is rotated to apply a centrifugal force to the reagent mixing chamber 40.

The solution in the reagent mixing chamber 40 flows through the first passage 51 and is transferred to the adsorption reaction chamber 20 by the centrifugal force of the disk 12.

After the solution is transferred to the adsorption reaction chamber 20, the disk 12 is repeatedly rotated forward/rearward through acceleration/deceleration, so that the adsorption medium 21 and the solution accommodated in the adsorption reaction chamber 20 are mixed with each other.

While the adsorption medium 21 is mixed with the solution, the nucleic acid in the solution is adsorbed to the adsorption medium 21, and the residual solution such as a reagent remaining after the nucleic acid is adsorbed remains in the adsorption reaction chamber 20.

In the present embodiment, a mixing time in the adsorption reaction chamber 20 may be 1 minute to 5 minutes.

When the mixing time is smaller than 1 minute, the nucleic acid is not properly adsorbed, and when the mixing time is larger than 5 minutes, there is no increase in an effect, and the specimen may be damaged.

When the mixing is completed, by the driver, the first valve 61 is operated to close the first passage 51 and the seventh valve 67 is operated to open the seventh passage 57.

Further, the disk 12 is rotated to apply a centrifugal force to the adsorption reaction chamber 20.

The residual solution in the adsorption reaction chamber 20 flows through the seventh passage 57 and is discharged to the wasted solution accommodating chamber 47 by the centrifugal force of the disk 12.

The above-described process may be repeated several times.

In the present embodiment, while the process is repeated three times, the residual solution is discharged after the nucleic acid is adsorbed (see FIG. 5D).

Next, the specimen, and the like remaining in the adsorption reaction chamber 20 are washed and removed.

In a state in which the residual solution is removed, the fifth valves 65 are operated to open the fifth passage 55.

The fifth passage 55 is opened, and the disk 12 is rotated to apply a centrifugal force to the washing liquid accommodating chambers 45.

The washing liquid in the washing liquid accommodating chambers 45 flows through the fifth passage 55 and is transferred to the adsorption reaction chamber 20 by the centrifugal force of the disk 12.

The washing liquid transferred to the adsorption reaction chamber 20 passes through the adsorption reaction chamber 20 and is discharged to the wasted solution accommodating chamber 47 through the seventh passage 57 by the centrifugal force of the disk 12.

In this process, while passing through the adsorption reaction chamber 20, the washing liquid washes the surface of the adsorption medium 21 and the inner surface of the adsorption reaction chamber 20.

When the washing liquid accommodating chambers 45 are provided in plurality, the fifth valves 65 connected with the washing liquid accommodating chambers 45 are sequentially operated to sequentially open the fifth passage 55, so that the washing operation is performed several times (see FIGS. 5E and 5F).

After the washing operation, the disk 12 is rotated to dry the adsorption medium 21.

The disk 12 is rotated to apply a centrifugal force to the adsorption reaction chamber 20.

All the residual solution such as the reagent remaining in the adsorption reaction chamber 20 flows to the wasted solution accommodating chamber 47 or is removed, by the centrifugal force of the disk 12.

In the present embodiment, the drying time may be 1 minute to 3 minutes.

When the drying time is smaller than 1 minute, the drying is not properly performed, and when the drying time is larger than 3 minutes, there is no increase in a drying effect, and the entire nucleic acid purification time is delayed.

When the drying is completed, the seventh valve 67 is operated to close the seventh passage 57.

Further, the sixth valve 66 is operated to open the sixth passage 56.

The sixth passage 56 is opened, and the disk 12 is rotated to apply a centrifugal force to the eluent accommodating chamber 46.

The eluent in the eluent accommodating chamber 46 flows through the sixth passage 56 and is transferred to the adsorption reaction chamber 20, by the centrifugal force of the disk 12.

After the eluent is transferred to the adsorption reaction chamber 20, the disk 12 is repeatedly rotated forward/rearward through acceleration/deceleration, so that the eluent and the adsorption medium 21 are mixed with each other.

While the adsorption medium 21 and the eluent are mixed with each other, the nucleic acid adsorbed to the adsorption medium 21 is eluted and is separated from the adsorption medium 21.

(see FIGS. 5G and 5H)

In the present embodiment, a mixing time during which the eluent is mixed in the adsorption reaction chamber 20 may be 30 seconds to 2 minutes. When the mixing time is smaller than 30 seconds, the nucleic acid is not properly separated, and when the mixing time is larger than 2 minutes, there is no increase in an effect anymore.

When the nucleic acid is completely separated by the eluent, the eighth valve 68 is finally operated to open the eighth passage 58.

The eighth passage 58 is opened, and the disk 12 is rotated to apply a centrifugal force to the adsorption reaction chamber 20.

The solution in the adsorption reaction chamber 20, that is, the solution including the nucleic acid separated from the adsorption medium 21, flows through the eighth passage 58 and is transferred to the nucleic acid solution accommodating chamber 48, by the centrifugal force of the disk 12 (see FIG. 51).

In this way, the entire process of purifying and extracting the nucleic acid from the specimen may be automated and may be integrally performed in a single disk 12.

### Experimental Example

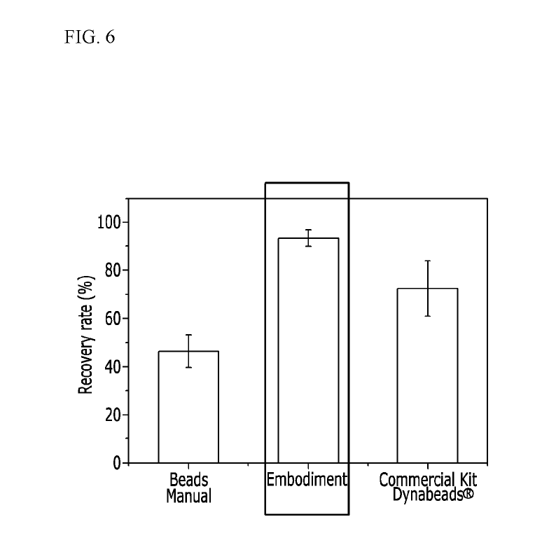

FIG. 6 is a graph depicting a result obtained by performing cell-free nucleic acid purification using the nucleic acid purification device according to the present embodiment.

The experiment was conducted by performing nucleic acid purification through the nucleic acid purification device according to the present embodiment by spiking 100 bp to 1000 bp of DNA ladders, which are an actual size range of the cell-free nucleic acid, in 350 μL of DNase-free water at a concentration of 1 ng/μL.

The total purification time according to the experiment is within 20 minutes, and a silica bead having a diameter of 100 μm is used as the adsorption medium.

In an experimental result, as illustrated in FIG. 6, when the nucleic acid purification is automated using the nucleic acid purification device according to the present embodiment, nucleic acid purification efficiency is further improved as compared with not only a case where the purification is performed manually but also a case where the purification is performed using the conventional commercialized kit.

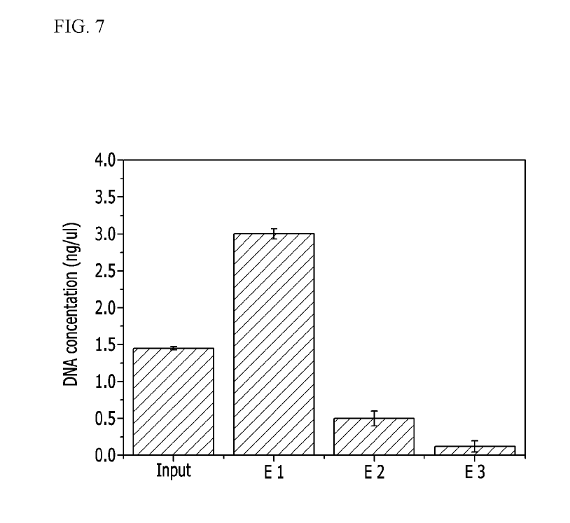

FIG. 7 is a graph depicting a result obtained by performing cell-free nucleic acid purification and concentration using the nucleic acid purification device according to the present embodiment.

In graph of FIG. 7, E1: First elution, E2: Second elution, and E3: Third elution represent an order of nucleic acid elution.

The experiment was conducted by performing purification through the nucleic acid purification device according to the present embodiment by spiking 100 bp to 1000 bp of DNA ladders, which are an actual size range of the cell-free nucleic acid, in 350 μL of DNase-free water at a concentration of 1.5 ng/μL (a concentration at input of graph of FIG. 7).

The total purification time according to the experiment is within 20 minutes, and a silica bead having a diameter of 100 μm is used as the adsorption medium.

In the experiment, the elution of the nucleic acid was performed three times in a sequence of E1, E2, and E3.

An experimental result indicates that the nucleic acid purified in the first elution (E1) is more concentrated than the first concentration at the input, and the nucleic acid remaining in the adsorption medium is eluted in the second elution (E2) and the third elution (E3).

It can be identified that the total amount of the nucleic acid purified through the three times of elution is larger than that of a commercialized product, as illustrated in FIG. 6. The device according to the present embodiment may be used to perform purification in a concentrated form according to use, and may be utilized for molecular diagnosis in a form suitable for each analysis method.

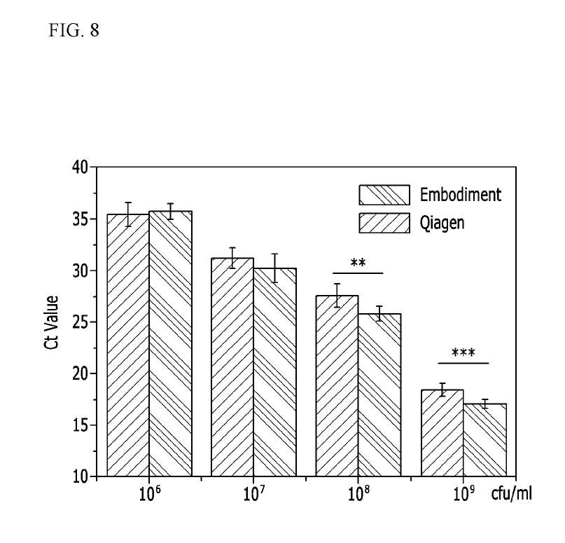

FIG. 8 is a graph depicting a result obtained by purifying a bacterial-derived nucleic acid (bacterial DNA(E. coli))DeletedTextsusing the nucleic acid purification device according to the present embodiment.

The experiment was conducted by extracting and purifying a bacterial-derived nucleic acid through the nucleic acid purification device according to the present embodiment by spiking a bacteria (E coli) in 200 μL of phosphate-buffered saline (PBS) at various concentrations.

The total purification time according to the experiment is within one hour, and the extracted and purified bacteria-derived nucleic acid (DNA) is quantified using an RT-PCR.

The experimental result indicates that as illustrated in FIG. 8, in the present embodiment, purification efficiency is higher than that of the commercialized product.

Thus, it is expected that the nucleic acid extracted and purified using the device according to the present embodiment may be used for various analysis methods for the molecular diagnosis.

Also, as mentioned above, it can be identified that the present device may be utilized for various targets by extracting and purifying the bacteria-derived nucleic acid as well as by purifying the cell-free nucleic acid.

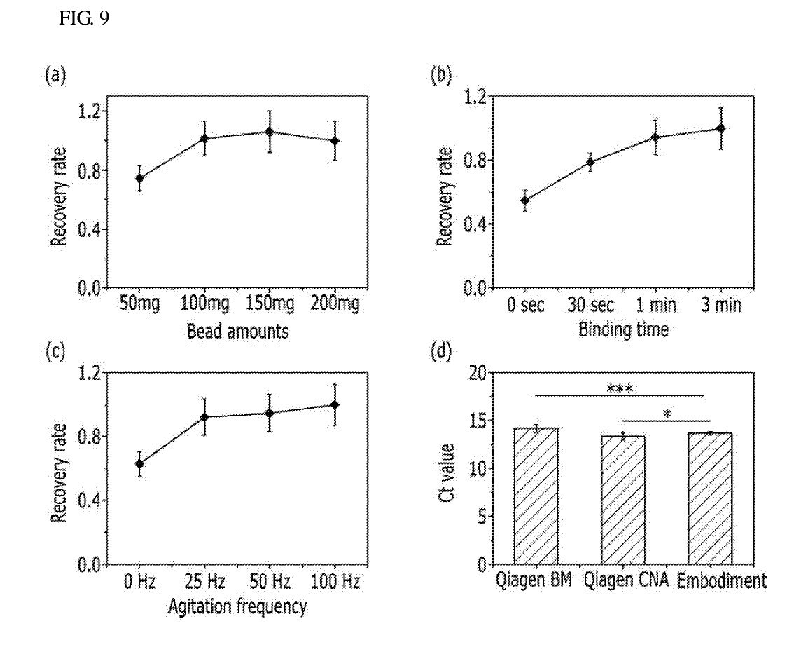

FIG. 9 illustrates a driving condition in an adsorption process as an example of various experimental results in which a driving condition for improving efficiency of the nucleic acid purification device according to the present embodiment is optimized.

The experiment was conducted by performing purification under various driving conditions through the nucleic acid purification device according to the present embodiment by spiking 300 bp of nucleic acids (DNAs), which are a size range of the cell-free nucleic acid, in 300 μL of human-derived materials (serum).

In the experimental result, as illustrated in FIGS. 9A, 9B, and 9C, optimum purification efficiency conditions for an amount, a mixing time (a binding time), and a mixing speed (an agitation frequency) of the silica beads which are nucleic acid adsorption mediums may be obtained.

Here, the mixing speed (the agitation frequency) means the number (Hz) of vibrations of repeated acceleration/deceleration performed during mixing reaction, for example, the number of vibrations of repeated acceleration/deceleration performed while the solution including the nucleic acid is mixed with the adsorption medium.

FIG. 9D illustrates a result obtained by comparing efficiency of the commercialized product with efficiency when 300 bp of nucleic acids (DNAs) are spiked in 300 μL of human-derived materials (serum) and purified while the nucleic acid purification device according to the present embodiment is driven under the optimized driving conditions according to the experimental result.

As illustrated in FIG. 9D, it can be identified that purification efficiency according to the present embodiment is higher than purification efficiency of the conventional commercialized product.

As in the experimental result, it can be identified that the nucleic acid may be actually purified, quantified, and analyzed from the human-derived materials using the present embodiment.

Although exemplary embodiments of the present invention have been described above, the present invention is not limited thereto. Further, it is apparent that various modifications may be conceived without departing from the scope of the appended claims, the detailed description of the invention, and the accompanying drawings, and may also belong to the scope of the present invention.

<Description of symbols>

| | |
|---|---|
| 10: Nucleic acid purification device | 12: Disk |
| 14: Rotary shaft | 16: Supply portion |
| 20: Adsorption reaction chamber | 21: Adsorption medium |
| 30: Solution accommodating portion | 31: Solution extraction port |
| 40: Reagent mixing chamber | 42: Separation chamber |

43: Enzyme accommodating chamber
44: Reinforcing agent accommodating chamber
45: Washing liquid accommodating chamber
46: Eluent accommodating chamber
47: Wasted solution accommodating chamber
48: Nucleic acid solution accommodating chamber
51 to 58: First passage to eighth passage
61 to 68: First valve to eighth valve
70: Driver

1. A nucleic acid purification device comprising:
a disk in which a fluid is transferred by a centrifugal force;
a supply portion installed in the disk and configured to supply a specimen and a reagent required for nucleic acid purification;
an adsorption reaction chamber which is installed in the disk and is connected with the supply portion and in which an adsorption medium for adsorbing a nucleic acid is accommodated and the nucleic acid is adsorbed from the specimen; and
a solution accommodating portion which is connected with an output side of an adsorption reaction portion along a centrifugal direction of the disk and in which a solution discharged through the adsorption reaction chamber by the centrifugal force is accommodated and is discharged to the outside.

2. The nucleic acid purification device of claim 1, the device further comprising:
a separation portion installed in the disk and connected with the adsorption reaction chamber to elute the nucleic acid adsorbed to the adsorption medium.

3. The nucleic acid purification device of claim 2, the device further comprising:
a washing portion installed in the disk and connected with the adsorption reaction chamber to supply a washing liquid to the adsorption reaction chamber.

4. A nucleic acid purification device comprising:
a disk in which a fluid is transferred by a centrifugal force;
a supply portion installed in the disk to supply a specimen and a reagent;
an adsorption reaction chamber which is installed in the disk and in which an adsorption medium is accommodated and a nucleic acid is adsorbed from the specimen supplied by the supply portion;
a washing portion installed in the disk to wash the adsorption reaction chamber;
a separation portion installed in the disk to elute the nucleic acid adsorbed to the adsorption medium;
a solution accommodating portion which is installed in the disk and in which a solution discharged from the adsorption reaction chamber is separately accommodated; and
a passage installed in the disk to control flow of the fluid moved according to the centrifugal force of the disk and a valve configured to selectively open/close the passage.

5. The nucleic acid purification device of claim 4, wherein
the valve configured to open/close the passage of the disk includes a blocking member installed on the passage of the disk, formed of an elastic material, and configured to open/close the passage while elastically deformed, a pressing member disposed outside the blocking member and configured to selectively open/close the passage by pressing the blocking member by an external force, and a support installed in the disk and supporting the pressing member.

6. The nucleic acid purification device of claim 5, the device further comprising:
a driver configured to selectively open/close the valve by applying an external force to the pressing member of the valve.

7. The nucleic acid purification device of claim 2, wherein
the separation portion includes an eluent accommodating chamber which is installed in the disk and in which an eluent is accommodated, a sixth passage which connects the eluent accommodating chamber and the adsorption reaction chamber and through which the eluent is transferred to the adsorption reaction chamber according to the centrifugal force of the disk, and a sixth valve configured to selectively open/close the sixth passage.

8. The nucleic acid purification device of claim 7, wherein
the washing portion includes a washing liquid accommodating chamber which is installed in the disk and in which a washing liquid is accommodated, a fifth passage which connects the washing liquid accommodating chamber and the adsorption reaction chamber and through which the washing liquid is transferred to the adsorption reaction chamber according to the centrifugal force of the disk, and a fifth valve configured to selectively open/close the fifth passage.

9-17. (canceled)

18. The nucleic acid purification device of claim 1, wherein
the supply portion, the adsorption reaction chamber, and the solution accommodating portion are sequentially arranged at a rotational center of the disk along a centrifugal direction such that the specimen flows along the supply portion, the adsorption reaction chamber, and the solution accommodating portion by the centrifugal force.

19. The nucleic acid purification device of claim 1, wherein
the adsorption medium is at least one selected from the group consisting of a bead, a column, and a post on a silica surface and a bead on a chitosan surface.

20. The nucleic acid purification device of claim 1, wherein
the specimen includes biofluid including blood, lymphatic fluid, tissue fluid, and urine or cells or small cells including somatic cells, bacteria, and viruses.

21. The nucleic acid purification device of claim 1, wherein
the adsorption reaction chamber has a gradient portion inclined such that a width of the gradient portion is narrowed toward an input side or an output side of the adsorption reaction chamber.

22. (canceled)

23. A nucleic acid purification method comprising:
a mounting step of mounting an adsorption medium for adsorbing a nucleic acid on an adsorption reaction chamber of a disk;
an injecting step of injecting a specimen into the disk;
an adsorbing step of adsorbing the nucleic acid by mixing the specimen with the adsorption medium by applying a centrifugal force to the adsorption reaction chamber;
a removing step of removing a solution remaining in the adsorption reaction chamber after the nucleic acid is adsorbed by applying the centrifugal force to the adsorption reaction chamber;
injecting an eluent into the adsorption reaction chamber; an eluting step of separating the nucleic acid from the adsorption medium by mixing the eluent with the adsorption medium by applying the centrifugal force to the adsorption reaction chamber; and
a discharging step of discharging a solution in which the nucleic acid is eluted from the adsorption reaction chamber by applying the centrifugal force to the adsorption reaction chamber.

24. The nucleic acid purification method of claim 23, wherein
the mounting step includes mounting a reagent for purifying the nucleic acid on the disk.

25. The nucleic acid purification method of claim 23, wherein
In the injecting step, the reagent for purifying the nucleic acid is mixed with the specimen, and a mixture of the specimen and the reagent is injected.

26. The nucleic acid purification method of claim 23, wherein
the adsorbing step includes opening a passage for transferring the solution, transferring the mixture to the adsorption reaction chamber by applying the centrifugal force by rotating the disk, and mixing the mixture with the adsorption medium by rotating the disk through acceleration/deceleration.

27. The nucleic acid purification method of claim 26, wherein
in the mixing of the mixture with the adsorption medium, a mixing time according to the rotation of the disk is 1 minute to 5 minutes.

28. The nucleic acid purification method of claim 26, wherein
the removing step includes opening a passage for transferring a remaining solution after the adsorption is performed in the adsorption reaction chamber, and discharging the residual solution to a wasted solution accommodating chamber by applying a centrifugal force by rotating the disk.

29-36. (canceled)

37. The nucleic acid purification method of claim 23, the method further comprising: a mixing step of mixing the specimen with a reagent for adsorbing the nucleic acid accommodated in a reagent mixing chamber, before the adsorbing step.

38. The nucleic acid purification method of claim 37, wherein
the mixing step includes opening a passage for transferring the specimen, transferring the specimen to the reagent mixing chamber by applying a centrifugal force by rotating the disk, closing the passage, and mixing the specimen with the reagent by rotating the disk through acceleration/deceleration.

39-45. (canceled)
